(19) United States
(12) Patent Application Publication
(10) Pub. No.: US 2008/0057144 A1
(43) Pub. Date: Mar. 6, 2008

(54) Wake up on time dietary supplement and method of use

(76) Inventor: Cathy Beggan, Sparta, NJ (US)

Correspondence Address: Arthur M. Peslak, Esq.;Mandel & Peslak, LLC, Suite 5, 80 Scenic Drive, Freehold, NJ, 07728-5211, US

(21) Appl. No.: 11/514,500

(22) Filed: Sep. 1, 2006

(51) Int. Cl. A61K 36/185; A61K 31/714; A61K 31/525; A61K 31/51

(52) U.S. Cl. 424/773; 514/251; 514/276; 514/350; 514/356; 514/567; 514/52

(57) ABSTRACT

A time-released dietary supplement is disclosed. The dietary supplement is meant be taken upon going to bed in the evening. The active ingredients are released during the night so the person will wake up energized and refreshed.

### BACKGROUND OF THE INVENTION

The present invention is directed to the field of dietary supplements in tablet or capsule form. In particular, the present invention is directed to the field of dietary supplements that allow the user to awaken in the morning refreshed and full of energy.

### SUMMARY OF THE INVENTION

The present invention is directed to a dietary supplement in tablet or capsule form that the user will take prior to going to bed. The tablet comprises an enteric exterior coating that will delay the release of the ingredients. As a result, the ingredients will be released into the person's system during the course of the night.

### DETAILED DESCRIPTION OF THE INVENTION

The present invention will now be described in terms of the presently contemplated preferred embodiment thereof. Those of ordinary skill in the art will recognize that many obvious changes may be made thereto without departing from the spirit or scope of the present invention.

The present invention is directed to the concept of a dietary supplement that is taken prior to going to bed at night. The preferred embodiment is a tablet or capsule formed with a time-release coating. The tablet slowly releases its ingredients into the user's bloodstream during the course of the night. The person will then wake-up naturally in the morning, feeling fully refreshed and full of energy due to the release of the ingredients while asleep.

The presently preferred embodiment of the invention includes ingredients that will physically energize the user as well as elevate his or her psychological mood. The active ingredients in the preferred embodiment are shown in Table 1.

TABLE 1

| | |
|---|---|
| B1 (Thiamin) | 75 mg |
| B2 (Riboflavin) | 75 mg |
| B3 (Niacin) | 75 mg |
| B6 | 75 mg |
| B12 | 1000 mg |
| Siberian Ginseng | 25 mg |
| (Eleuthero Root Extract) | |
| Guarana Seed Extract | 300 mg |
| L-Tyrosine | 300 mg |

It is contemplated that Panax Ginseng may be substituted for Siberian Ginseng. In addition, the alternative embodiments of the formulation may also include the following:

| | | |
|---|---|---|
| Vitamin A | 5000 | |
| Vitamin C | 300 | mg |
| Vitamin E | 200 | |
| Selenium | 200 | mcg. |

The ingredients of the preferred embodiment are manufactured in tablet form. The tablet is provided with an enteric coating. The enteric coating is the agent, which controls the release of the ingredients into the user's bloodstream over the course of the nights sleep. It is contemplated that the user will take the tablet of the present invention in the conventional manner with a glass of water prior to going to sleep.

Those of ordinary skill in the art will recognize the foregoing merely describes one embodiment of the present invention and that there many obvious changes may be made thereto without departing from the spirit or the scope of the present invention as forth in the appended claims.

What is claimed is:

1) A method for waking up in an energized state in the morning comprising:
a) providing a time release dietary supplement comprising active ingredients adapted to energize the user upon waking but not including any sleep inducing ingredients;
b) taking the time release dietary supplement prior to falling asleep at night; and;
c) releasing the active ingredients from the time release dietary supplement into the user's bloodstream over the course of the night while the user sleeps.

2) The method of claim 1 whereby the dietary supplement comprises a tablet with an enteric coating.

3) The method of claim 1 whereby the active ingredients comprise thiamin in the approximate amount of 75 mg.

4) The method of claim 1 whereby the active ingredients comprise riboflavin in the approximate amount of 75 mg.

5) The method of claim 1 whereby the active ingredients comprise Niacin in the approximate amount of 75 mg.

6) The method of claim 1 whereby the active ingredients comprise B6 in the approximate amount of 75 mg;

7) The method of claim 1 whereby the active ingredients comprise B12 in the approximate amount of 1000 mg.

8) The method of claim 1 whereby the active ingredients comprise eleuthero root extract in the approximate amount of 25 mg.

9) The method of claim 1 whereby the active ingredients comprise guarana root extract in the approximate amount of 300 mg.

10) The method of claim 1 whereby the active ingredients comprise L-Tyrosine in the approximate amount of 300 mg.

11) A dietary supplement for use in waking up in an energized state comprising:
a) active ingredients adapted so that the user will wake up energized and refreshed; and
b) a time release mechanism adapted so that the user can take the supplement prior to bed and the active ingredients will be released during the course of the night into the user's bloodstream.

12) The dietary supplement of claim 11 whereby the supplement is manufactured in tablet form and the time-release mechanism is an enteric coating on an exterior surface of the tablet.

13) The dietary supplement of claim 11 whereby the active ingredients comprise Thiamin.

14) The dietary supplement of claim 11 whereby the active ingredients comprise Niacin.

15) The dietary supplement of claim 11 whereby the active ingredients comprise B6.

16) The dietary supplement claim 11 whereby the active ingredients comprise B12.

17) The dietary supplement of claim 11 whereby the active ingredients comprise eleuthero root extract.

18) The dietary supplement of claim 11 whereby the active ingredients comprise guarana seed extract.

19) The dietary supplement of claim 11 whereby the active ingredients comprise L-Tyrosine.

20) A dietary supplement in tablet form for waking up energized in the morning comprising.
a) active ingredients adapted so that the user will wake up energized and refreshed; and
b) a time released coating.

21) The dietary supplement claim 20 wherein the active ingredients comprise Thiamin, riboflavin, Niacin, B6, B12, eleuthero root extract, guarana seed extract and L-Tyrosine.

22) The method of claim 2 wherein the active ingredients in the dietary supplement comprise Thiamin in the approximate amount of 75 mg, riboflavin in the approximate amount of 75 mg, Niacin in the approximate amount of 75 mg, B6 in the approximate amount of 75 mg, B12 in the approximate amount of 1000 mg, eleuthero root in the approximate amount of 25 mg, guarana root in the approximate amount of 300 mg and L-Tyrosine in the approximate amount of 300 mg.
